(19) United States
(12) Patent Application Publication
(10) Pub. No.: US 2026/0276088 A1
(43) Pub. Date: Sep. 17, 2026

(54) TUBE CLAMPING DEVICE AND OPERATING METHOD

(71) Applicant: Kendrion Kuhnke Automation GmbH, Malente (DE)

(72) Inventors: Lukas NUPPENAU, Bargteheide (DE); Inga DOHSE, Malente (DE); Burghart SCHNABEL, Kiel (DE); Volker KUEHN, Schacht-Audorf (DE); Jochen STEGELMANN, Gronenberg (DE); Jannes PAAP, Kiel (DE)

(73) Assignee: Kendrion Kuhnke Automation GmbH, Malente (DE)

(21) Appl. No.: 19/569,199

(22) Filed: Mar. 17, 2026

(30) Foreign Application Priority Data
Mar. 17, 2025 (EP) 25 164 241.9

(51) Int. Cl. F16K 7/04; F16K 31/06; F16K 31/44

(52) U.S. Cl. CPC F16K 7/045; F16K 31/0675; F16K 31/44; F16K 2200/305

(57) ABSTRACT

A tube clamping device including: a housing; and a tube feed-through arranged outside the housing and reversible in its cross-section between a clamping position and an open position. Wherein the tube feed-through is formed by a fixed counter bearing and a pressure piece movable relative to the fixed counter bearing and is connected to a plunger of an electromagnet that is spring-loaded in a direction of the clamping position and moves the pressure piece away from the counter bearing against a spring load when the electromagnet is energized and thereby moves the tube feed-through from the clamping position to the open position, and wherein the pressure piece is connected to the plunger via an adapter, by which a gap width between the pressure piece and the fixed counter bearing assumed in the clamping position of the tube feed-through can be adjusted.

### CROSS-REFERENCE TO RELATED APPLICATION

The present application is based upon and claims the benefit of priority from EP 25 164 241.9 filed on Mar. 17, 2025, the entire contents of which is incorporated herein by reference.

### BACKGROUND

### Field

The present disclosure relates to a tube clamping device with a housing and a tube feed-through arranged outside the housing and reversible in its cross-section between a clamping position and an open position, which is formed by a fixed counter bearing and a pressure piece that can be moved relative to the fixed counter bearing and is connected to a plunger of an electromagnet that is spring-loaded in the direction of the clamping position and moves the pressure piece away from the counter bearing against the spring load when the electromagnet is energized and thereby moves the tube feed-through from the clamping position to the open position. The present disclosure also relates to a method for operating a tube clamping device.

### Prior Art

Tube clamping devices for clamping tubes are known, in particular in medical applications such as dialysis. Such tube clamping devices have an electromagnet that is activated to open a feed-through opening for a tube, known as a tube feed-through, or to close it to such an extent that any flow of liquids or gases through the tube is clamped off. To do this, a pressure piece with a wedge-shaped edge is usually moved towards a fixed counter bearing with a defined clamping force until a minimum gap height of the clamping gap of the tube feed-through is reached. Since the tube itself must not be damaged, there are tube clamping devices with different dimensions for different tube diameters. Several such tube clamping devices for different tube sizes can be installed in a device so that only a small part with the respective tube feed-through protrudes outwards and is accessible, while the part with the electromagnet is located inside the device.

The operation of a tube clamping device essentially has three operating states, namely tube insertion, normal operation and emergency operation. To insert the tube, the tube feed-through is opened by energizing the solenoid coil, pulling the plunger into the solenoid coil and thereby moving the pressure piece rigidly connected to the plunger, which opens the clamping gap. In normal operation, the tube is inserted into the tube feed-through and the solenoid coil is energized when fluid is to flow through the tube and de-energized when the tube is to be clamped. In the event of a malfunction, e.g. a power failure, the standard state should be that the tube is clamped. For this reason, the tube clamping device is always configured so that an electric current through the solenoid coil of the electromagnet is required to open it. However, this means that in an emergency, when no power is available to energize the solenoid coil, the tube is clamped by default. This results in emergency operation, in which the tube can be removed even in the event of a power failure by an emergency release mechanism in which the pressure piece is actuated mechanically or manually to open the clamping gap.

There are various solutions for securing the tube in the tube feed-throughs of known tube clamping devices. There are tube clamping devices with completely enclosed tube feed-throughs. These provide a secure hold for the tubes and prevent them from slipping out of the tube feed-through when open. However, the tubes have to be threaded through such a tube feed-through, which is not always easy.

There are also tube feed-throughs that are open on one side, which make it very easy to insert the tube. However, with these tube feed-throughs, there is a risk that the tube may slip out sideways when the feed-through is open, for example if it is under mechanical tension. Some tube guides that are open on one side have a semicircular recess in the contour of the counter bearing opposite the pressure piece, which can hold the tube in place, whereby the size of the recess is dimensioned for the type of tube for which the tube clamping device is intended.

In addition to securing tubes against slipping out sideways, some of the problems encountered with known tube feed-throughs also stem from the need to have a large number of tube clamping devices available for different tube diameters. The pressure pieces of these tube clamping devices each have constant stroke lengths and tend to jam, which contributes to a reduction in service life.

### SUMMARY

The present disclosure provides a tube clamping device and a method for operating it that overcome some of the disadvantages of known tube clamping devices.

This task can be accomplished by a tube clamping device with a housing and a tube feed-through arranged outside the housing and reversible in its cross-section between a clamping position and an open position, which is formed by a fixed counter bearing and a pressure piece that can be moved relative to the fixed counter bearing and is connected to a plunger of an electromagnet that is spring-loaded in the direction of the clamping position and moves the pressure piece away from the counter bearing against the spring load when the electromagnet is energized and thereby moves the tube feed-through from the clamping position to the open position, wherein the pressure piece is connected to the plunger via an adapter, by which a gap width between the pressure piece and the fixed counter bearing assumed in the clamping position of the tube feed-through can be adjusted.

The adapter can make it possible to adjust the clamping gap and thus to adjust and use the tube clamping device for different tube sizes. This can make it possible to keep a smaller quantity of tube clamping devices in stock than before, because it is no longer necessary to use separate tube clamping devices for each tube size, which have to be kept in stock. The adapter can change the distance between the pressure piece and the plunger, so that the clamping gap can be changed while the stroke length of the plunger remains the same. This can be achieved, for example, by a screw-nut connection wherein a nut, on which a support body is placed that supports the pressure piece, can be adjusted in its position on the screw that is connected to the plunger. Clamping connections can also be used in which a retaining element can be moved on a bolt connected to the plunger.

In embodiments of the tube clamping device, the adapter can be adjustable when the tube clamping device is in its assembled state and can be accessible through an opening in the housing. The adjustment can be made, for example, in such a way that the adapter can be connected to the plunger of the electromagnet via a screw connection and can be adjusted in its distance from the plunger by rotating it relative to the plunger. In embodiments, the adapter can be gripped and held through an opening in the housing by a suitable instrument, and at the same time the plunger can be rotated about its axis with a lower part of the bolt, so that the distance of the non-rotating adapter from the plunger increases or decreases. The adapter may also be configured and arranged so that it is prevented from rotating with the bolt by a shape-complementary part inside the housing. In this case, no opening in the housing is necessary through which the adapter is gripped. Conversely, in other embodiments, the adapter may also be rotated while the plunger remains in its position. The adjustment can be made as the final assembly step, which significantly reduces tooling and development costs, but subsequent modification is also possible. The clamping gap size can thus be adjusted to a new tube size without having to remove the tube clamping device from the device in which it is mounted. This can ensure adequate hygiene.

In a further embodiment, the pressure piece can be flexibly connected to the plunger and/or decoupled from the plunger. This can result in a tolerant configuration which, in the event of a possible misadjustment of the clamping gap with respect to a tube diameter, can dissipate excessive forces so that the tube is not damaged. The flexible connection can also prevent damage caused by the pressure piece jamming during the stroke. The connection of the pressure piece to the plunger can, for example, be configured such that it is made via one or more intermediate pieces which absorb excessive forces in a non-destructive manner, for example in an elastic manner, by reversible deformation, or which undergo inelastic deformation when a limit force is exceeded, thereby ensuring that the excessive force is kept away from the tube. In one embodiment, an additional elastic element may be located on the circumference of the adapter, which allows misalignment between the adapter and the pressure piece. The force transmission for clamping a tube in the clamping gap can be achieved by a form fit between the adapter and the pressure piece, even in the event of misalignment, so that the required forces can be transmitted reliably. The return stroke when opening the gap can then be achieved by the elastic element on the circumference of the adapter.

In embodiments, the fixed counter bearing on one side of the tube feed-through can be connected to the housing and can have a lateral opening on the other side for threading in or removing a tube to be clamped, whereby a push button can be arranged on the open side of the tube feed-through, which can close the lateral opening of the tube feed-through in a rest state and releases it when pressed by a stroke length. This can address the problem that a recess in the fixed counter bearing can only provide reliable protection against lateral slippage out of the tube feed-through for tubes of a certain diameter. Instead, the side opening can be reversibly closed by the push button.

To insert a tube into the tube guide, the electromagnet can be energized so that the pressure piece moves away from the fixed counter bearing and opens the clamping gap. In addition, the push button can be pressed in until the side opening of the tube guide is released and the tube can then be inserted into the tube guide from the side. Once the tube has been inserted, the push button can be released again so that it returns to its rest position, in which it closes the side opening of the tube guide. The tube can be now prevented from slipping out of the tube feed-through from the side. In addition, the power supply to the electromagnet can be switched off so that the pressure piece clamps the tube and prevents any fluid from flowing through it.

In a further refinement that allows emergency operation of the tube clamping device, the push button can be connected to a lever arranged inside the housing at a first end of the lever moving with the stroke of the push button, while a second end of the lever can be connected to a joint in the housing, wherein, when the push button is actuated and the electromagnet is de-energized, the lever can contact the plunger directly or indirectly and presses against the spring preload of the plunger in order to move the tube feed-through into the open position. This makes it possible to use the push button not only to release the side opening, but also to open the clamping gap in the emergency mode if the electromagnet is de-energized. Since this counteracts the spring preload of the plunger to which the pressure piece is connected, this further movement of the push button can require a greater force than that required in normal operation to release the side opening.

In embodiments, the push button can have a width in the direction of the lateral extension of the tube feed-through that is greater than the maximum distance between a clamping contact side of the pressure piece and a clamping contact side of the fixed counter bearing opposite the clamping contact side of the pressure piece. This wide configuration can make it possible to operate the push button manually while leaving a distance between the hand and the lateral opening of the tube feed-through that allows a tube to be removed from or inserted into the tube feed-through.

The push button can be spring-loaded by a return spring into a position closing the lateral opening of the tube feed-through.

In embodiments, the spring preload of the plunger can be produced by a clamping spring arranged on the side of the plunger facing away from the pressure piece. The clamping spring can be supported on a counter bearing attached to the solenoid coil or the yoke of the electromagnet. The clamping spring can be configured, for example, as a coil spring or compression spring.

The object can also be achieved by a method for operating a tube clamping device described above, wherein a minimum size of a gap between the pressure piece and the fixed counter bearing of the tube clamping device can be set by the adapter. This method can achieve the same advantages, properties and features as the tube clamping device of the present disclosure.

In a further development, the push button can be actuated to release the side opening of the tube feed-through and released to close the side opening, and can be pressed further than is necessary to release the side opening of the tube feed-through for emergency opening of the tube feed-through.

Further features are apparent from the description of embodiments together with the claims and the accompanying drawings. Embodiments may fulfil individual features or a combination of several features.

### BRIEF DESCRIPTION OF THE DRAWINGS

Embodiments are described below without limiting the general concept of the invention, using embodiments with reference to the drawings, whereby express reference is made to the drawings for all details not explained in detail in the text. In the Drawings:

FIG. 1 illustrates a known tube clamping device in schematic perspective view,

FIG. 2 illustrates the tube clamping device from FIG. 1 in cross-section,

FIG. 3 illustrates a schematic perspective view of an embodiment of a tube clamping device according to the present disclosure,

FIG. 4 illustrates the tube clamping device from FIG. 3 in cross-section,

FIG. 5 illustrates details of the tube clamping device in a perspective view,

FIG. 6 illustrates central components of the tube clamping device in cross-section, and

FIG. 7 illustrates a perspective view of an exemplary embodiment of an adapter.

In the drawings, identical or similar elements and/or parts are marked with the same reference numbers, so that they need not be introduced again.

### DETAILED DESCRIPTION

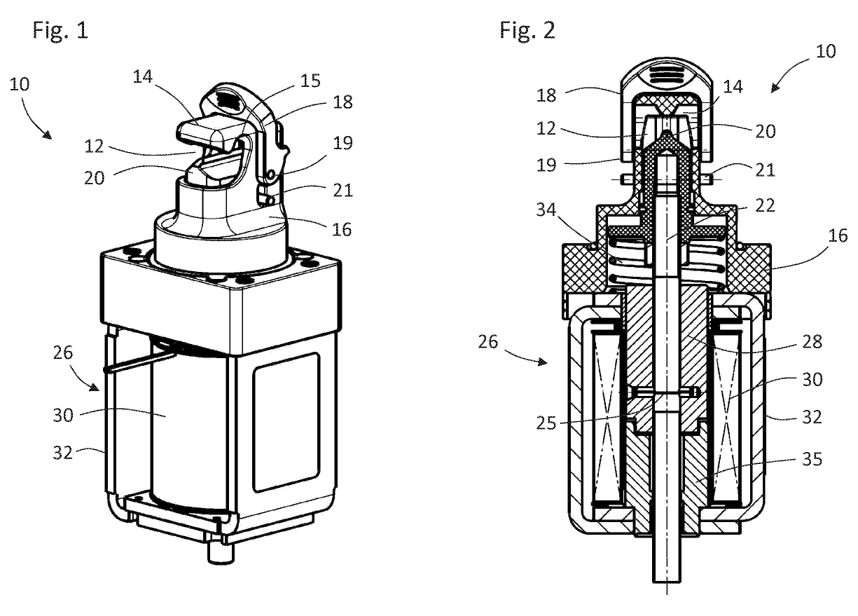

FIG. 1 shows a known tube clamping device 10 for a specific tube size in a schematic perspective view. At its tip, it has a tube feed-through 12, which is bounded on one side by a movable pressure piece 20 with a wedge-shaped profile and on the other by a counter bearing 14. The counter bearing 14 is connected to one side of the tube feed-through 12 with a housing 16 of the tube clamping device 10 or is made in one piece with the housing 16. The tube feed-through 12 is open on one side so that a tube (not shown) can be inserted into or removed from the tube feed-through 12. Furthermore, the tube feed-through 12 has a semicircular recess 15 on the underside of the counter bearing 14, which serves to prevent the tube from slipping out of the tube feed-through 12 sideways through the opening. The recess 15 is adapted to the diameter of the type of tube for which the tube clamping device 10 is intended.

Furthermore, the tube clamping device 10 has an emergency release lever 18, which is mounted on the housing 16 so that it can be tilted on a hinge 19. In an emergency, the emergency release lever 18 is folded back. The side opening of the tube feed-through 12 always remains open. The lower part of the emergency release lever 18 is equipped with an eccentric slide which, when the emergency release lever 18 is actuated, contacts a drive pin 21 connected to the pressure piece 20. When further actuated, the eccentric slide pushes down the drive pin 21 and the pressure piece 20 connected to it in order to open the clamping gap forming the tube feed-through 12. A tube positioned in the tube feed-through 12 can then be removed.

If the tube clamping device 10 is installed in a device with several tube clamping devices, the upper part of the tube clamping device 10 protrudes from the device. The larger lower part is concealed in the device and is not accessible from the outside. It comprises a further part of the housing 16 and an electromagnet 26 that serves to move the pressure piece 20 from the clamping position to an open position. The solenoid coil 30 and yoke 32 of the electromagnet 26 are shown in FIG. 1.

FIG. 2 shows details of the tube clamping device 10 from FIG. 1 in cross-section. The wedge-shaped cross-section of the pressure piece 20 is clearly visible, as is the fact that in this embodiment, the counter bearing 14 is also essentially wedge-shaped in cross-section. The pressure piece 20 has a flat extension on its underside, against which a clamping spring 34 presses from below, which is supported on the yoke 32 of the electromagnet 26.

The pressure piece 20 is firmly connected to the plunger 28 of the electromagnet 26 via a rigid push rod 22. In its upper region, the push rod 22 has a transverse drive pin 21, which is part of the emergency release system described in connection with FIG. 1. When the eccentric slide in the lower region of the emergency release lever 18 hits the drive pin 21, it pushes it downwards and thereby moves the push rod 22 and the pressure piece 20 downwards, opening the tube feed-through 12.

FIG. 2 also shows that the electromagnet 26 has, from the outside to the inside, a yoke 32, the solenoid coil 30 and the plunger 28 as central components. Furthermore, there is a bearing 35 inside the solenoid coil 32, which provides a lower stop and thus limits the penetration depth of the plunger 28 into the solenoid coil 30. FIG. 2 also shows that the plunger 28 is connected to the rigid push rod 22 via a split pin 25.

The state shown in FIGS. 1 and 2 is the state in which the solenoid coil 30 is energized and pulls the plunger 28 into the solenoid coil 30 until it stops on the bearing 35. The pressure piece 20 is pulled along via the rigid push rod 22, thus opening the tube feed-through 12. This occurs against the force exerted by the clamping spring 34 on the underside of the pressure piece 20. As soon as the solenoid coil 30 is no longer energized, the spring force of the clamping spring 34 no longer counteracts this force and the clamping spring 34 pushes the pressure piece 20 upwards with its defined spring force in order to reduce the clamping gap of the tube feed-through 12. This stroke is limited by the fact that the upper side of the flat extension on the underside of the pressure piece 20 comes into contact with the upper side of the housing 16. These limiting surfaces and the stop at the bearing 35 define the stroke that the pressure piece 20 of the tube clamping device 10 performs between the clamping position and the open position.

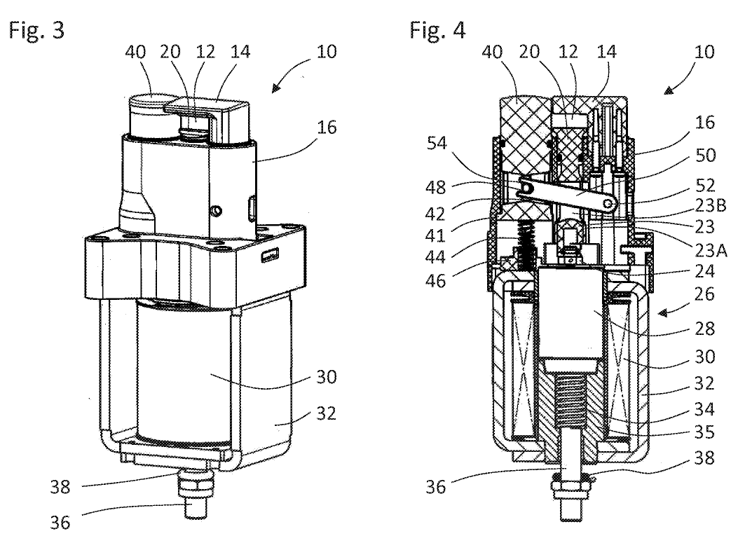

FIG. 3 shows an example of a tube clamping device 10 according to the present disclosure in a schematic perspective view. The most obvious external differences to the known tube clamping device 10 from FIG. 1 are found in the area of the tube feed-through 12. While the pressure piece 20 is similar in structure to that of FIG. 1 in its upper part, the counter bearing 14 in this case forms an "L"-shaped part without a semicircular recess. One leg of the "L" of the counter bearing 14, which is directly opposite the pressure piece 20, forms the upper boundary of the tube feed-through 12. This part is also roughly wedge-shaped in cross-section. The other leg of the counter bearing 14 forms the connection to the housing 16 and a lateral boundary of the tube feed-through 12.

On the other side, the tube feed-through 12 is open. It is bounded by a push button 40, which closes the open side end of the tube feed-through 12. The push button 40 can be actuated by applying pressure to the surface of the push button 40. The push button 40 then sinks into the housing 16 and releases the lateral opening of the tube feed-through 12. In this embodiment, the push button 40 is configured to be so wide that it can be touched at its end facing away from the counter bearing 14, leaving a free space through which a tube can be inserted into or removed from the tube feed-through 12 from the side.

FIG. 4 shows a cross-section of the tube clamping device 10 from FIG. 3. Here, further differences to the known tube feed-through 10 from FIGS. 1 and 2 become apparent. In the tube feed-through 10 in FIGS. 3 and 4, the electromagnets 26 are constructed similarly to those in the other forms in FIGS. 1 and 2, but in this case the plunger 28 on the underside opposite the bearing 35 is spring-loaded with a clamping spring 34, which pushes the plunger 28 upwards out of the solenoid coil 30 when the solenoid coil 30 is not energized. When energized, the plunger 28 is pulled into the solenoid coil 30 until it contacts the stop, i.e. the top of the bearing 35.

A bolt 36 engages with the plunger 28, the lower end of which is provided with a nut and an annular end bearing damper 38, which forms a stop for the upward movement of the plunger 28 in the clamping direction of the tube clamping device 10. The bolt 36 is firmly connected to the plunger 28, for example by a split pin or a screw. When the lower end of the bolt 36 is turned, the plunger 28 turns with it. The adapter 24 is connected to the plunger 28 by a screw connection. To adjust the clamping gap, the adapter 24 can be held in place by a suitable instrument, which is inserted through an opening in the housing 16 for this purpose, while at the same time the distance between the adapter 24 and the plunger 28 is changed by turning the bolt 36.

Further differences can be seen in the upper part inside the housing 16. The push button 40 is centrally connected to a bolt 42, which is immersed in a return spring 44 on its underside, which preloads the push button 40 with a spring force that pushes the push button 40 into its upper position, in which the tube feed-through 12 is closed at the side. The return spring 44 presses against a bearing 46, which also serves as a stop for the underside of the bolt 42. As soon as the underside of the bolt 42 touches this stop, the push button 40 can no longer be pressed down further. An upper stop is provided by a rim 41 on the underside of the push button 40, which, in an upper position of the push button 40, abuts against a circumferential edge in the housing 16.

The bolt 42 also has a transverse driver 48, which moves with the bolt 42 or the push button 40. One end of a lever 50 rests on this driver 48 with an elongated groove 54. The lever 50 is pivotably mounted on a joint 52 at its other end on the opposite side. When the push button 40 is actuated, the driver 48 thus takes the lever 50 with it, which is pivoted about the joint 52.

This pivoting of the lever 50 causes the lever 50 to contact a first connecting part 23A of the connection between the plunger 28 and the pressure piece 20 and to exert a force on the push button 40. This means that, as soon as sufficient force is applied, the plunger 28 and thus also the pressure piece 20 are pressed downwards, counteracting the spring force of the clamping spring 34. This implements an emergency opening function. The activation of the emergency opening function can also be clearly felt because pressing down the push button 40 initially only acts against the restoring force of the restoring spring 44, but once the top of the connecting piece 23 is contacted, it also acts against the force provided by the clamping spring 34, which is significantly greater than the restoring force of the restoring spring 44.

Unlike in the device shown in FIGS. 1 and 2, the connection between the plunger 28 and the pressure piece 20 is not rigid. Instead, an adapter 24 is mounted on the plunger 28, which is accessible from the outside even when the tube clamping device 10 is installed and with which the clamping gap, i.e., the size of the tube feed-through 12 in the clamping position, can be adjusted. The adapter 44 may, for example, be a clamping device on a bolt or a nut with an internal thread on a bolt with an external thread, which is connected to the plunger 28.

In the embodiment, a connecting piece 23 sits on the adapter 24, which is formed integrally with the pressure piece 20 and comprises the first and second connecting parts 23A, 23B. The connecting piece 23 has a passage for the lever 50 and can be formed with spring elements to provide additional flexibility. However, it can also consist of separate connecting parts 23A, 23B that sit on top of each other, thus providing flexibility. The lever 50 presses on the top of the first connecting part 23A or the connecting part 23B when the emergency opening function is activated. The gap between the first connecting part 23A and the pressure piece 20 is bridged by a second connecting part 23B, which forms the passage for the lever 50. This can be formed with spring elements, which, on the one hand, leave an opening for the passage of the lever 50 and, on the other hand, introduce flexibility into the connection between the plunger 28 and the pressure piece 20. The second connecting piece 23B can thus be configured such that it bends when an upper pressure limit is exceeded, thereby absorbing the excess force that could damage a tube, which can be the case if the adapter 24 is not correctly adjusted to the size of a currently applied tube and the clamping gap becomes too small. In this way, the inserted tube can be further protected.

In this embodiment of a tube clamping device 10, the three operating states of tube insertion, normal operation and emergency operation are implemented in such a way that, in order to insert the tube, the clamping gap of the tube feed-through 12 is opened by energizing the solenoid coil 30 of the electromagnet 26, whereby the plunger 26 performs a stroke against the force of the clamping spring 34. By manually pressing the push button 40 against the return spring 44 of the push button 40, the tube feed-through 12 is released and the tube can be inserted sideways into the tube feed-through 12. As soon as the push button 40 is no longer pressed, it moves to its upper end position and prevents the tube from accidentally falling out of the tube feed-through 12.

During normal operation, the clamping gap of the tube pass-through 12 between the pressure piece 20 and the counter bearing 14 is opened and closed according to the desired application, thereby opening or clamping the inserted tube. Opening is achieved by energizing the solenoid coil 30, which exerts a force on the plunger 28, causing it to perform the stroke in conjunction with the pressure piece 20. Closing is a safety-related function. Closing is performed mechanically in the de-energized state via the clamping spring 34 and is therefore independent of the power supply. The end position damping 38 reduces the noise level. The pressure piece 20 is mechanically decoupled from the plunger 28 via the adapter 24 and the connecting parts 23A, 23B. The special mechanical decoupling can compensate for misalignments of the pressure piece 20, prevent overdetermined bearing conditions, reduce friction and significantly extend the service life of the tube clamping device 10.

In the event of a power failure, the emergency mode is activated. The plunger 28 drops and the tube is securely clamped between the counter bearing 14 and the pressure piece 20 by the small spring 34. If the tube is to be removed in this state, the resulting force can be transferred to the pressure piece 20 via the lever 50 by applying increased manual force to the push button 40. The tube clamping device 10 opens. Furthermore, the side opening of the tube feed-through 12 opens and the tube can be removed.

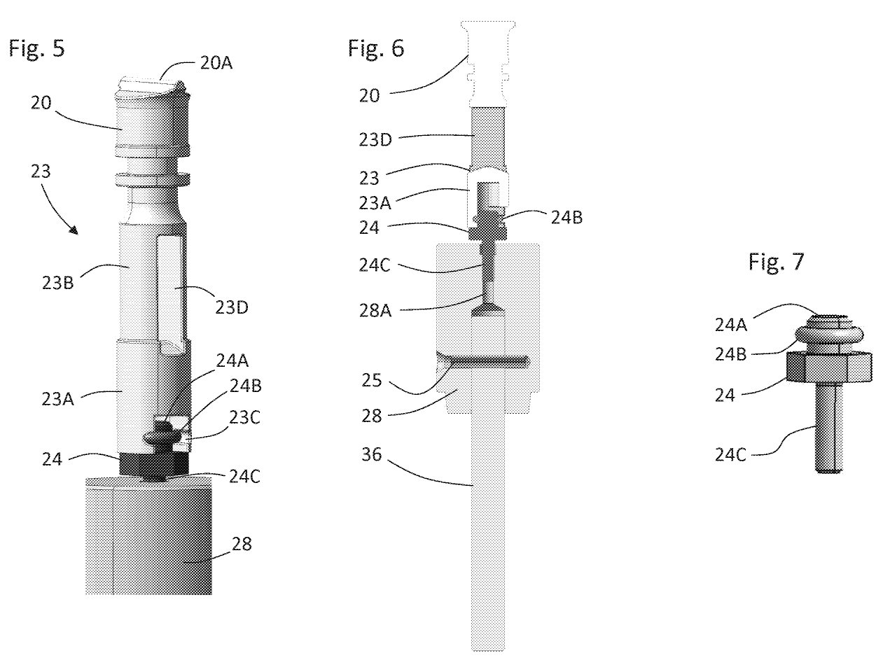

FIG. 5 shows details of the connection between the plunger 28 and the pressure piece 20 in a perspective view. The adapter 24 is located on the plunger 28, the base of which in the present embodiment has the shape of a hexagon, essentially like a screw nut. Downwardly, the adapter 24 has an elongated lower adapter part 24C with an external thread (not shown), which is screwed into a corresponding opening with an internal thread in the upper side of the plunger 28. By turning the adapter 24 relative to the plunger 28, the distance between the base of the adapter 24 and the plunger 28 can be changed and adjusted.

The connecting piece 23 sits on top of the base of the adapter 24. In this embodiment, the connecting piece 23 is formed integrally with the pressure piece 20, which has a clamping wedge 20A on its upper side for clamping a tube. The connecting piece 23 is elongated and has several sections along its length. Above the adapter 24 is a first connecting part 23A, which has an annular inner groove 23C parallel to its lower edge. The connecting piece 23 sits on the base of the adapter 24 with its lower part. The adapter 24 has a short upper adapter part 24A on its upper side, which has an O-ring 24B on an annular groove (not shown), which engages on its outer side in the annular inner groove 23C of the first connecting part 23A. The O-ring 24B transmits the tensile force to the connecting piece 23 when the solenoid coil 30 of the electromagnet 26 is energized and the plunger 24 is pulled back to open the clamping gap. When the clamping gap is closed, the base of the adapter 24 presses directly onto the underside of the connecting piece 23. A second connecting piece 23B between the first connecting piece 23A and the pressure piece 20 has a through-opening 23D through which the lever 50 shown in FIG. 4 is guided.

FIG. 6 shows a cross-section of some of the central components of the tube clamping device 10. Here it can be seen that the bolt 36 is connected to the plunger 28 of the electromagnet via a split pin 25. The bolt 36 extends approximately halfway into the plunger 28. Above the bolt, the receptacle that accommodates the bolt 36 in its lower part narrows to the diameter of the lower adapter part 24C of the adapter 24 and has an internal thread that matches the external thread on the lower adapter part 24C. The adapter 24 is thus screwed into the receptacle in the plunger 28 and can be adjusted in its distance from the plunger 28 by screwing it in and out to adjust the size of the clamping gap. For this purpose, either the adapter 24 itself or the plunger 28 can be rotated about its axis.

A perspective view of the adapter 24 with the O-ring 24B extended on the upper adapter part 24A is shown schematically in FIG. 7. The hexagonal shape of the base of the adapter 24 is suitable for being gripped and held, for example, by a wrench when the plunger 28 is rotated to adjust the distance between the base of the adapter 24 and the plunger 28.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCE SIGNS

10 Tube clamping device

12 Tube feed-through

14 Counter bearing

15 Recess

16 Housing

18 Emergency release lever with eccentric

19 Hinge

20 Pressure piece

20A Clamping wedge

21 Drive pin

22 Push rod

23 Connecting piece

23A First connecting piece

23B second connecting part

23C ring-shaped inner groove

23D through opening

24 Adapter

24A Upper adapter part

24B O-ring

24C Lower adapter part

25 Split pin

26 Electromagnet

28 Armature

28A Central bore

30 Solenoid coil

32 Yoke

34 Clamping spring

35 Bearing

36 Bolt

38 End position damping

40 Push button

41 Edge

42 Bolt

44 Return spring

46 Bearing

48 Driver

50 Lever

52 Joint

54 Groove

What is claimed is:

1. A tube clamping device comprising:
a housing; and
a tube feed-through arranged outside the housing and reversible in its cross-section between a clamping position and an open position,
wherein the tube feed-through is formed by a fixed counter bearing and a pressure piece movable relative to the fixed counter bearing and is connected to a plunger of an electromagnet that is spring-loaded in a direction of the clamping position and moves the pressure piece away from the counter bearing against a spring load when the electromagnet is energized and thereby moves the tube feed-through from the clamping position to the open position, and
wherein the pressure piece is connected to the plunger via an adapter , by which a gap width between the pressure piece and the fixed counter bearing assumed in the clamping position of the tube feed-through can be adjusted.

2. The tube clamping device according to claim 1, wherein the adapter is adjustable when the tube clamping device is in its assembled state and is accessible through an opening in the housing.

3. The tube clamping device according to claim 1, wherein the pressure piece is one or more of flexibly connected to the plunger and is decoupled from the plunger.

4. The tube clamping device according to claim 1, wherein
the fixed counter bearing is connected to the housing on one side of the tube feed-through and has a lateral opening on an other side for threading in or removing a tube to be clamped, and
a push button is arranged on an open side of the tube feed-through which closes the lateral opening of the tube feed-through in a rest state and releases it when pressed by a stroke length.

5. The tube clamping device according to claim 4, wherein
the push button is connected to a lever arranged inside the housing at a first end of the lever moving with the stroke of the push button, while a second end of the lever is connected to a joint in the housing, and
when the push button is actuated and the electromagnet is de-energized, the lever contacts the plunger directly or indirectly and presses against the spring preload of the plunger in order to move the tube feed-through into the open position.

6. The tube clamping device according to claim 4, wherein the push button has a width in the direction of a lateral extension of the tube feed-through that is greater than a maximum distance between a clamping contact side of the pressure piece and a clamping contact side of the fixed counter bearing opposite the clamping contact side of the push piece.

7. The tube clamping device according to claim 4, wherein the push button is spring-loaded by a return spring into a position closing the lateral opening of the tube feed-through.

8. The tube clamping device according to claim 1, wherein the spring preload of the plunger is produced by a clamping spring arranged on the side of the plunger facing away from the pressure piece.

9. A method for operating a tube clamping device comprising:
a housing; and
a tube feed-through arranged outside the housing and reversible in its cross-section between a clamping position and an open position,
wherein the tube feed-through is formed by a fixed counter bearing and a pressure piece movable relative to the fixed counter bearing and is connected to a plunger of an electromagnet that is spring-loaded in a direction of the clamping position and moves the pressure piece away from the counter bearing against a spring load when the electromagnet is energized and thereby moves the tube feed-through from the clamping position to the open position, and
wherein the pressure piece is connected to the plunger via an adapter, by which a gap width between the pressure piece and the fixed counter bearing assumed in the clamping position of the tube feed-through can be adjusted;
the method comprising, setting a minimum size of the gap between the pressure piece and the fixed counter bearing of the tube clamping device by the adapter.

10. The method according to claim 9, further comprising actuating the push button to release the lateral opening of the tube feed-through and released to close the lateral opening.

11. The method according to claim 10, wherein, for emergency opening of the tube feed-through, the push button is actuated further than is necessary to release the lateral opening of the tube feed-through.
